(19) United States
(12) Patent Application Publication
(10) Pub. No.: US 2008/0077130 A1
(43) Pub. Date: Mar. 27, 2008

(54) High Frequency Incision Tool For Endoscope

(75) Inventors: Hiroaki SHIBATA, Saitama (JP); Takaaki TATEBAYASHI, Tokyo (JP); Yoshinori MORITA, Hyogo (JP)

Correspondence Address: GREENBLUM & BERNSTEIN, P.L.C., 1950 ROLAND CLARKE PLACE, RESTON, VA, 20191, US

(73) Assignees: PENTAX Corporation, Tokyo (JP); Kobe University, Hyogo (JP)

(21) Appl. No.: 11/859,905

(22) Filed: Sep. 24, 2007

(30) Foreign Application Priority Data
Sep. 25, 2006 (JP) 2006-258317

(51) Int. Cl. A61B 18/14

(52) U.S. Cl. 606/46; 600/104

(57) ABSTRACT

A high frequency incision tool for an endoscope includes a flexible insulating tube configured to be inserted into and pulled out of a treatment tool insertion channel of the endoscope, a conductive wire connectable with a high frequency power supply, the conductive wire being inserted and arranged in the flexible insulating tube, a protruded portion formed to be partially protruded forward at a distal end portion of the flexible insulating tube, the protruded portion having a rounded leading edge, and a high frequency electrode provided as a portion of the conductive wire that is exposed out of the flexible insulating tube, the high frequency electrode including at least a portion, closest to the leading edge, which is substantially perpendicular to a virtual plane including therein an axis line of the flexible insulating tube and the leading edge.

### BACKGROUND OF THE INVENTION

The present invention relates to a high frequency incision tool that makes it possible to securely perform a high frequency incision treatment.

As a high frequency incision tool for an endoscope for incising an elevated portion of surface mucosa in a body cavity by high frequency cautery, conventionally, there has been used an incision tool with a high frequency electrode formed in a linear or curved rod shape being arranged to protrude forward from a distal end of a flexible tube or an incision tool with a conductive wire as a high frequency electrode being crossly arranged at a leading edge portion of a front hood of an endoscope (e.g., Japanese Patent Provisional Publications No. 2002-153485 and No. 2005-66140).

When incising the elevated portion of the surface mucosa in the body cavity, it is desired from a standpoint of security to cauterize and incise only a mucosa region without cauterizing a muscle layer beneath the mucosa region.

However, a conventional high frequency incision tool has a problem that its distal end portion with the high frequency electrode arranged thereon might gradually approach the muscle layer beneath the mucosa region contrary to an operator's intention and cause a region around the muscle layer to be cauterized and damaged. This is because the high frequency incision tool, which is being pushed in a cautery treatment, is easy to advance toward the cauterized tissue with less resistance against the advance of the high frequency incision tool due to a region of cauterized tissue evenly spreading around the high frequency electrode.

### SUMMARY OF THE INVENTION

The present invention is advantageous in that there can be provided an improved high frequency incision tool for endoscope, of which a distal end is hard to approach a muscle layer beneath a mucosa region contrary to an operator's intention when the operator pushes the tool while cauterizing an elevated portion of the surface mucosa so that the operator can securely perform a high frequency incision treatment.

According to an aspect of the present invention, there is provided a high frequency incision tool for an endoscope, which includes a flexible insulating tube configured to be inserted into and pulled out of a treatment tool insertion channel of the endoscope, a conductive wire connectable with a high frequency power supply, the conductive wire being inserted and arranged in the flexible insulating tube, a protruded portion formed to be partially protruded forward at a distal end portion of the flexible insulating tube, the protruded portion having a rounded leading edge, and a high frequency electrode provided as a portion of the conductive wire that is exposed out of the flexible insulating tube, the high frequency electrode including at least a portion, closest to the leading edge of the protruded portion, which is substantially perpendicular to a virtual plane including therein an axis line of the flexible insulating tube and the leading edge of the protruded portion.

Optionally, the high frequency electrode may be arranged at a first half portion of the distal end portion of the flexible insulating tube opposite a second half portion that includes the rounded leading edge of the protruded portion when viewed from a front side of the flexible insulating tube.

Optionally, the protruded portion may be formed in a slantwise-cut shape with the rounded leading edge.

Optionally, the protruded portion may be formed to partially protrude in a tongue shape with the rounded leading edge.

Optionally, the high frequency incision tool may further include a pair of openings arranged in a circumferential direction on an outer circumferential surface of the distal end portion of the flexible insulating tube, the pair of openings being configured such that the conductive wire can run therethrough.

Yet optionally, the high frequency electrode may be exposed out of the flexible insulating tube between the pair of openings and arranged along an outer circumferential surface of the first half portion of the distal end portion of the flexible insulating tube.

Alternatively, the high frequency electrode may be exposed out of the flexible insulating tube between the pair of openings and arranged along a distal end surface of the distal end portion of the flexible insulating tube.

Further optionally, the pair of openings may be formed symmetrically with respect to the virtual plane.

Optionally, the high frequency electrode may be configured to be substantially perpendicular to the virtual plane over an entire length thereof.

Alternatively, the high frequency electrode may be formed substantially U-shaped, the U-shaped electrode including a pair of first portions substantially parallel to the virtual plane and a second portion substantially perpendicular to the virtual plane between the pair of first portions.

Optionally, the flexible insulating tube may include a first tube and a second tube. Further optionally, the first tube may be connected with the second tube to be rotatable around the axis line of the flexible insulating tube with respect to the second tube.

### BRIEF DESCRIPTION OF THE ACCOMPANYING DRAWINGS

FIG. 1 is a cross-sectional side view of a distal end portion of a high frequency incision tool for an endoscope in a first embodiment according to the present invention.

FIG. 2 is a top view of the high frequency incision tool in the first embodiment according to the present invention.

FIG. 3 is a front view of the high frequency incision tool in the first embodiment according to the present invention.

FIG. 4 is a side view showing an entire configuration of the high frequency incision tool in the first embodiment according to the present invention.

FIG. 5 is a side view showing the entire configuration of the high frequency incision tool in an operated state in the first embodiment according to the present invention.

FIG. 6 is a cross-sectional top view of the distal end portion of the high frequency incision tool in an operating state in the first embodiment according to the present invention.

FIGS. 7 to 10 are illustrations for sequentially showing states where a percutaneous endoscopic treatment of incising an elevated mucosa region is performed using the high frequency incision tool in the first embodiment according to the present invention.

FIG. 11 is a cross-sectional top view of a distal end portion of a high frequency incision tool for an endoscope in a second embodiment according to the present invention.

FIG. 12 is a side view of the distal end portion of the high frequency incision tool in the second embodiment according to the present invention.

FIG. 13 is a front view of the distal end portion of the high frequency incision tool in the second embodiment according to the present invention.

FIG. 14 is a top view of a distal end portion of a high frequency incision tool for an endoscope in a third embodiment according to the present invention.

FIG. 15 is a side view of the distal end portion of the high frequency incision tool in the third embodiment according to the present invention.

FIG. 16 is a front view of the distal end portion of the high frequency incision tool in the third embodiment according to the present invention.

FIG. 17 is a side view of a distal end portion of a high frequency incision tool for an endoscope in a fourth embodiment according to the present invention.

### DETAILED DESCRIPTION OF THE EMBODIMENTS

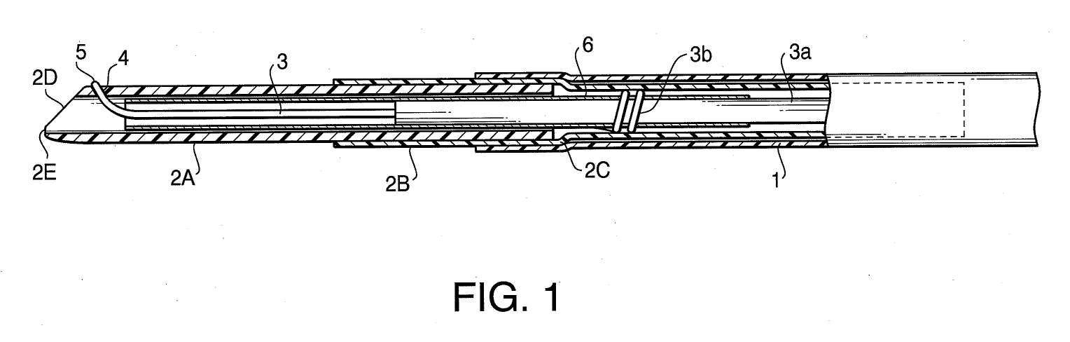

Referring to the accompanying drawings, embodiments of the present invention will be described. FIGS. 1, 2 and 3 are a cross-sectional side view, a top view, and a front view showing a distal end portion of a high frequency incision tool for an endoscope in a first embodiment according to the present invention, respectively.

Reference numbers 1, 2A, and 2B of FIG. 1 represent a flexible tube with a diameter of about 2 mm to be inserted into and pulled out of a treatment tool insertion channel (not shown) of the endoscope, for example, which is made of an electric insulating synthetic resin such as an ethylene tetrafluoride resin.

A rear tube 1 of the flexible tube is a longer tube with an entire length of about 1 to 2 m. Meanwhile, each front tube 2A or 2B of the flexible tube is a shorter tube with an entire length of several centimeters. The front tubes 2A and 2B are laminated and integrally conjugated at a joint portion thereof to form a stopper step 2C to be tightly pressed into a distal end of the rear tube 1. Accordingly, the front tubes 2A and 2B may be formed as a thermoformed single tube.

The front tubes 2A and 2B are loosely inserted and fitted in the rear tube 1 at a rear portion (right side in FIG. 1) from the stopper step 2C. Therefore, in a state where the stopper step 2C is not pressed into the distal end of the rear tube 1, the front tubes 2A and 2B can slide back and forth along and rotate around an axis line of the flexible tube 1, 2A, and 2B with respect to the rear tube 1. Meanwhile, at a state where the stopper step 2C is pressed into the distal end of the rear tube 1, the front tubes 2A and 2B are fixed to the rear tube 1 with a certain degree of strength.

The rear tube 1 is formed to have a constant diameter over an entire length thereof. However, the rear tube 1 is elastically deformed with the diameter thereof being enlarged at the distal end portion thereof into which the stopper step 2C of the front tubes 2A and 2B is pressed.

There is inserted and arranged in the flexible tube 1, 2A, and 2B over the entire length thereof a flexible conductive wire 3 to be connected with a high frequency power supply at a rear end side of the flexible tube 1, 2A, and 2B in a state slidable back and forth along and rotatable around the axis line of the flexible tube 1, 2A, and 2B. A reference number 3a represents a covering tube for the high frequency electrode 3.

A distal end of the front tube 2A is formed with a part protruded forward. In the first embodiment, a distal end surface 2D of the front tube 2A is formed in a slantwise-cut shape with a rounded leading edge portion 2E.

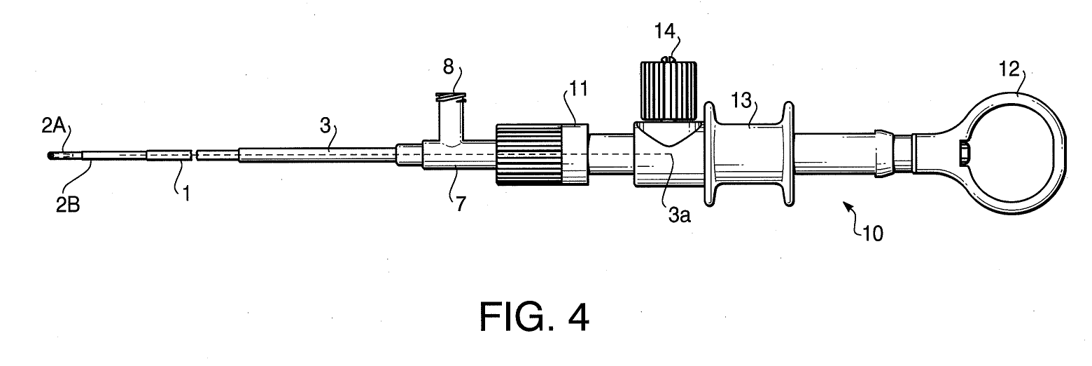

It is noted that, as shown in FIG. 3, a location of the leading edge portion 2E of the front tube 2A when viewed from a front side of the front tube 2A is referred to as a lower side of the front tube 2A. An alternate long and short dash line X represents a virtual plane including therein the axis line of the front tube 2A and the leading edge portion 2E.

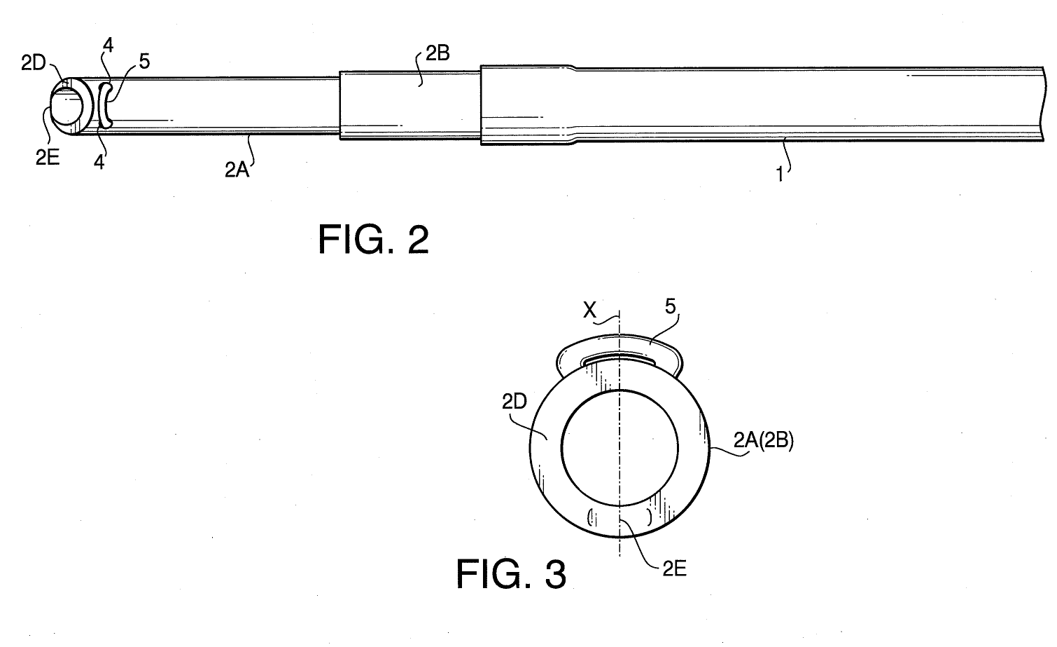

As shown in FIGS. 1 and 2, in an area adjacent to the distal end surface 2D on an upper outer circumferential surface of the distal end portion of the front tube 2A, a pair of openings 4 configured such that the conductive wire 3 can run therethrough are provided symmetrically with respect to the virtual plane X. The conductive wire 3 is exposed along the outer circumferential surface of the front tube 2A between the pair of openings 4.

A portion of the conductive wire 3 exposed on the outer circumferential surface between the pair of openings 4 serves as a high frequency electrode 5 for high frequency cautery. Accordingly, as shown in FIG. 3, the high frequency electrode 5 is not arranged on a lower outer circumferential surface (that is, on a side close to the leading edge portion 2E) of the front tube 2A. Further, the high frequency electrode 5 is arranged to be substantially perpendicular to the virtual plane X.

As shown in FIG. 1, an end portion 3b of the conductive wire 3 housed back into the front tubes 2A and 2B through the pair of openings 4 is wounded and fixed around an insulating tube 6 covering the conductive wire 3.

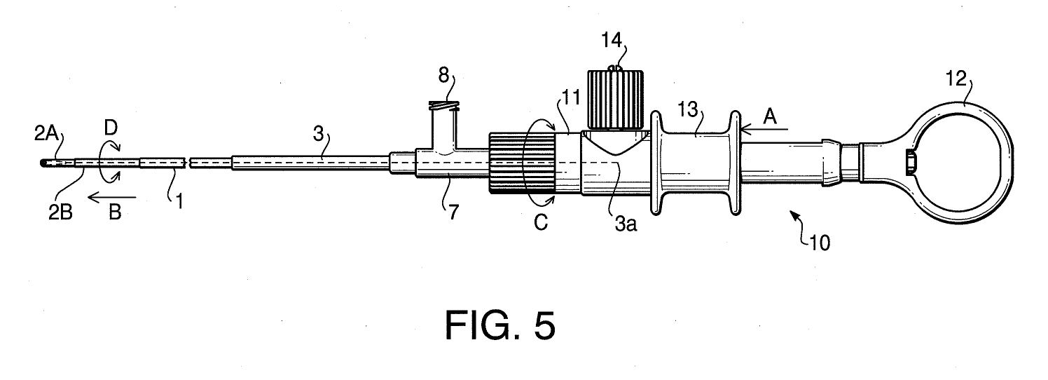

FIG. 4 shows an entire configuration of the high frequency incision tool for an endoscope. As shown in FIG. 4, a rear end pipe sleeve 7 fixed to a rear end of the rear tube 1 has a liquid supplying pipe sleeve 8 formed to be protruded sideward and connectable with a syringe. By supplying cleaning liquid from the liquid supplying pipe sleeve 7, it is possible to squirt the supplied liquid out of an opening of the distal end of the front tube 2A via a void of the flexible tube 1, 2A, and 2B.

The rear end pipe sleeve 7 is linked with an operating unit 10. The operating unit 10 has a fixed finger-operating portion 12 attached to an end portion at a hand side of an operating unit body 11 connected with the rear end pipe sleeve 7 to be rotatable around the axis line with respect to the rear end pipe sleeve 7. In addition, the operating unit 10 has a slidable finger-operating portion 13 attached to the operating unit body 11 to be slidable back and forth along the axis line with respect to the operating unit body 11.

A rear end 3a of the conductive wire 3 is linked and fixed to the slidable finger-operating portion 13. Further, a connection terminal 14 to be connected with a high frequency power supply cord (not shown) is attached to the slidable finger-operating portion 13. Hence, by connecting the high frequency power supply cord with the connection terminal 14, a high frequency current can be conveyed to the high frequency electrode 5 via the conductive wire 3.

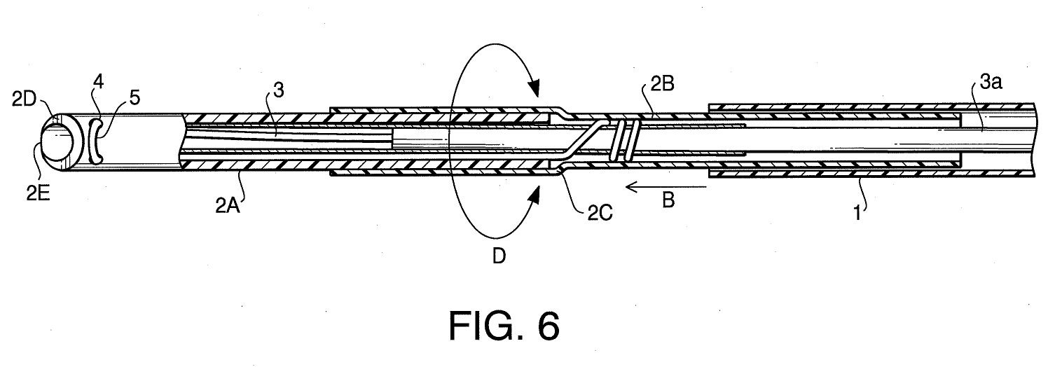

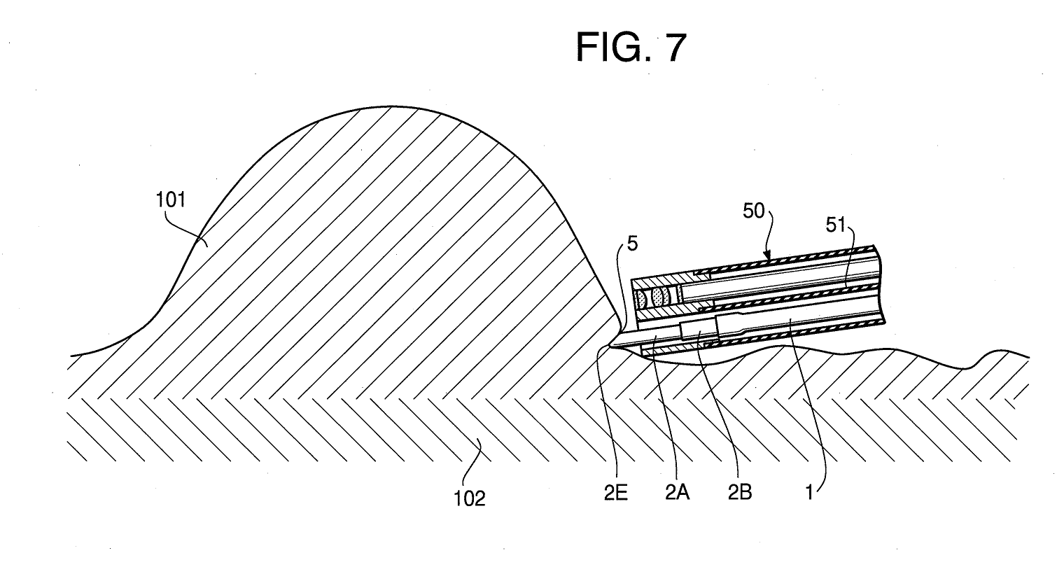

In the aforementioned configuration, when the slidable finger operating portion 13 is pushed forward as indicated by an arrow A in FIG. 5, the stopper step 2C of the front tubes 2A and 2B is pushed forward out of the inside of the rear tube 1 as indicated by an arrow B in FIGS. 5 and 6 such that the front tubes 2A and 2B come into a free state, namely, a state where the front tubes 2A and 2B are not fixed to the rear tube 1.

In this state, as indicated by an arrow C in FIG. 5, by rotating the entire operation unit 10 around the axis line with respect to the rear end pipe sleeve 7 as indicated by an arrow C in FIG. 5, the front tubes 2A and 2B are rotated around the axis line with respect to the rear tube 1 as indicated by an arrow D in FIGS. 5 and 6. Thereby, it is possible to arbitrarily adjust a positional relationship in the rotational direction between the rear tube 1 and the high frequency electrode 5. After the adjustment is completed, the stopper step 2C is pressed into the rear tube 1 again such that the front tubes 2A and 2B are fixed to the rear tube 1.

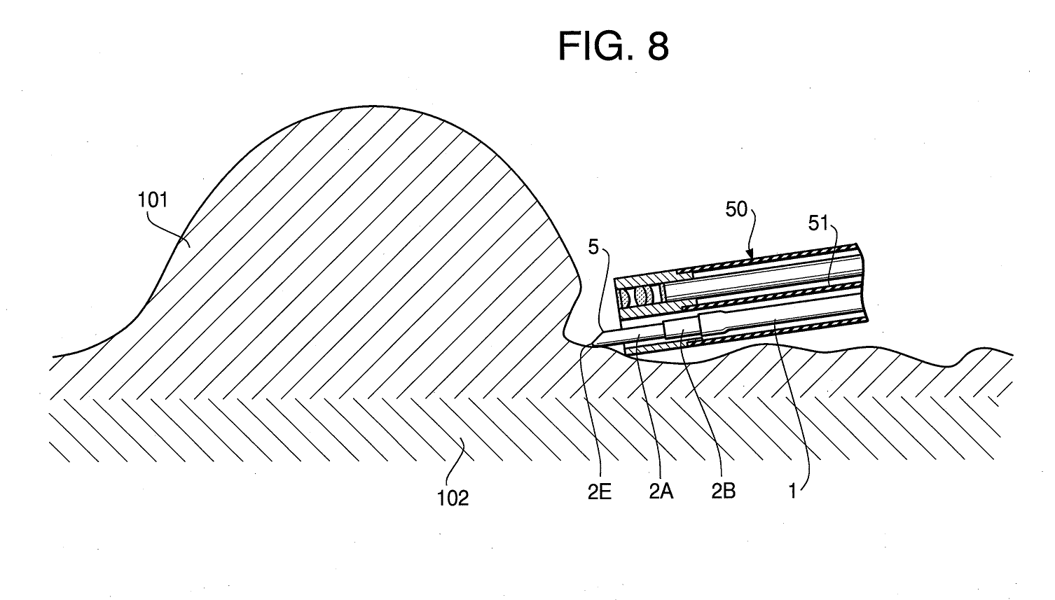

FIGS. 7 to 10 show sequential states where a percutaneous endoscopic treatment of incising an elevated mucosa region 101 is performed using the high frequency incision tool for an endoscope configured as above in the first embodiment. As shown in FIG. 7, firstly, the leading edge portion 2E of the flexible tube 1, 2A, and 2B inserted into the treatment tool insertion channel 51 is pushed onto a region close to a base of the elevated mucosa region 101. At this time, the position of the front tubes 2A and 2B in the rotational direction has previously been adjusted such that the leading edge portion 2E is located at a side closer to a muscle layer 102 while the high frequency electrode 5 is located at a side farther from the muscle layer 102.

Then, when a high frequency current is conveyed to the high frequency electrode 5, as shown in FIG. 8, tissue of the elevated mucosa region 101 around a region contacting with the high frequency electrode 5 is cauterized and incised. Meanwhile, tissue around a region closer to the muscle layer 102 than the leading edge portion 2E is hardly cauterized since it is away from the high frequency electrode 5.

Subsequently, as shown in FIG. 9, when the flexible tube 1, 2A, and 2B is pressed forward, the leading edge portion 2E is advanced along the tissue. However, it can be prevented that the leading edge portion 2E approaches the muscle layer 102 contrary to an operator's intention, since the tissue around the region closer to the muscle layer 102 than the leading edge portion 2E is not cauterized.

Then, when the high frequent current is conveyed to the high frequency electrode 5 again, as shown in FIG. 10, tissue of the elevated mucosa region 101 around a region contacting with the high frequency electrode 5 is cauterized and incised, and the elevated mucosa region 101 is securely incised around the base thereof without a cauterized region approaching to a side of the muscle layer 102.

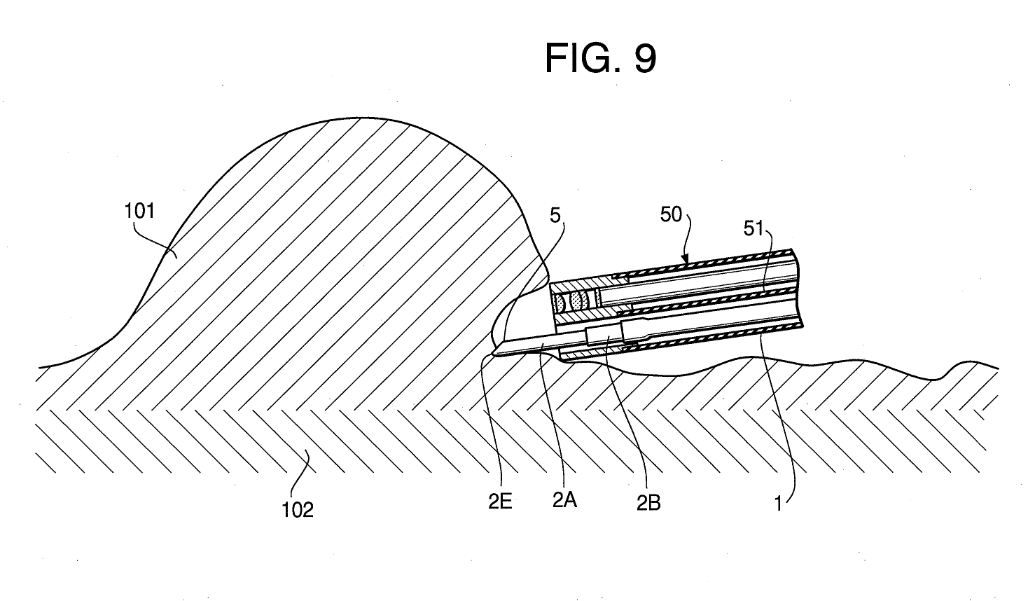

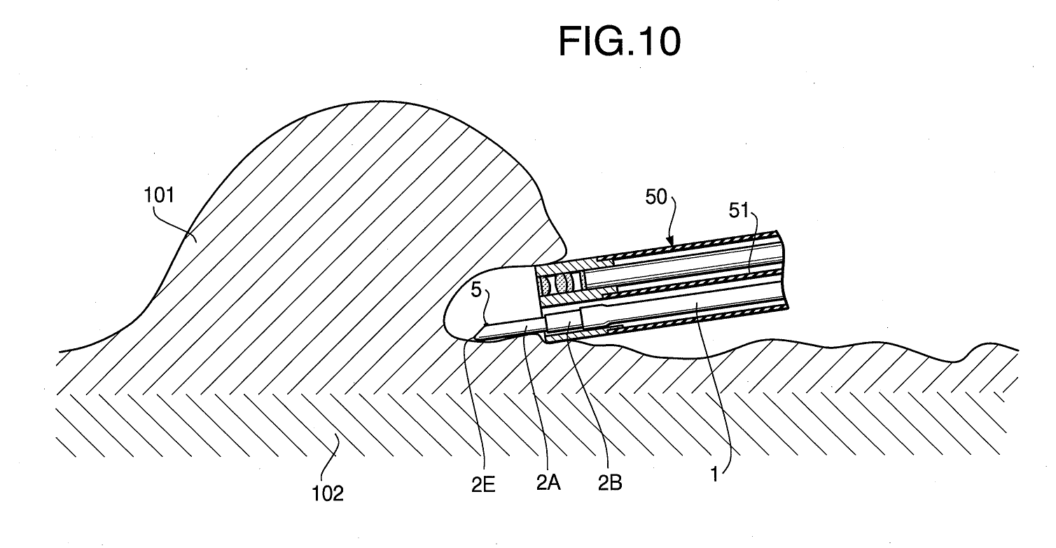

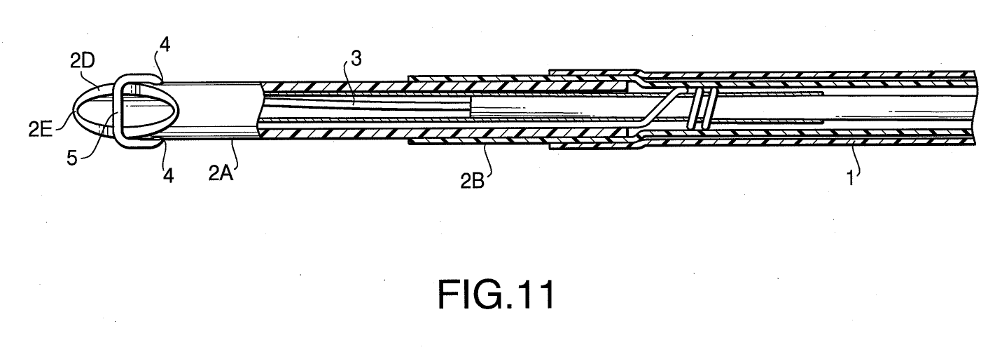

FIGS. 11 to 13 show a cross-sectional top view, a side view, and a front view of a distal end portion of a high frequency incision tool for an endoscope in a second embodiment according to the present invention, respectively. As shown in FIGS. 11 to 13, a high frequency electrode 5 is exposed on a distal end surface 2D of a front tube 2A between a pair of openings 4 and arranged to be substantially perpendicular to the virtual plane X over an entire length thereof. Such configuration brings the same effects as the first embodiment.

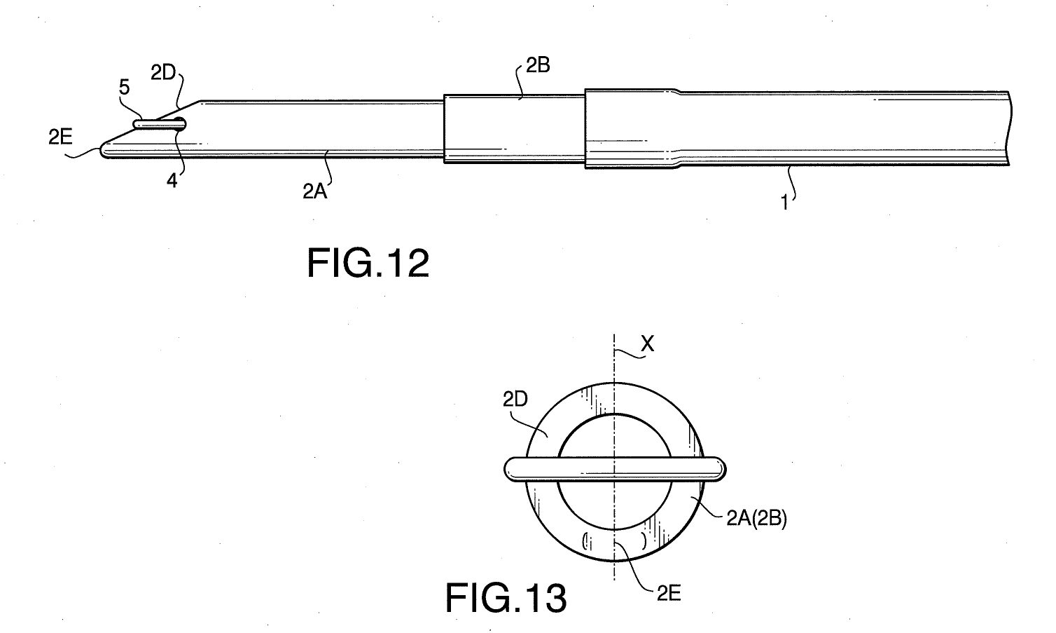

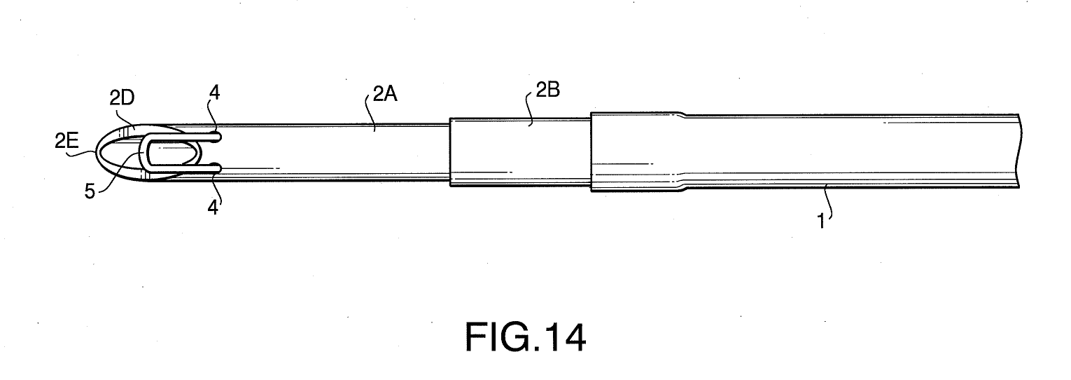

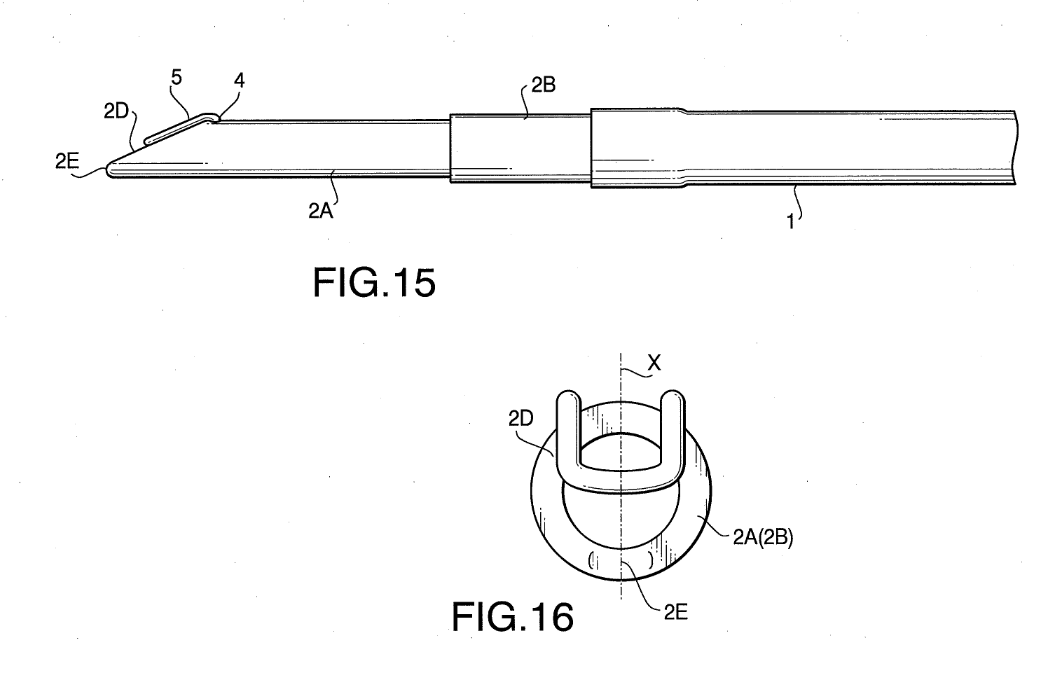

FIGS. 14 to 16 show a top view, a side view, and a front view of a distal end portion of a high frequency incision tool for an endoscope in a third embodiment according to the present invention, respectively. As shown in FIGS. 14 to 16, a high frequency electrode 5 is formed substantially U-shaped and located such that a pair of parallel portions of the U-shaped electrode 5 are arranged along a distal end surface 2D to be parallel to the virtual plane X and such that a bridge portion between the parallel portions of the U-shaped electrode 5 is substantially perpendicular to the virtual plane X. Such configuration brings the same effects as the first embodiment.

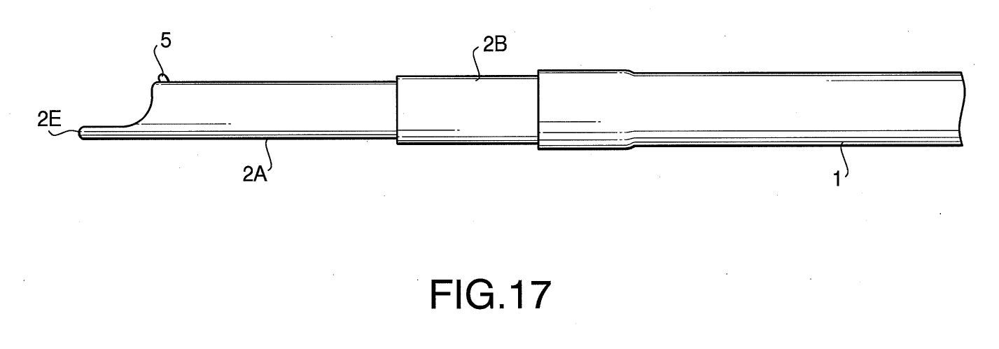

FIG. 17 is a side view of a distal end portion of a high frequency incision tool for an endoscope in a fourth embodiment according to the present invention. As shown in FIG. 17, a front tube 2A has a distal end portion formed to partially protrude in a tongue shape with a rounded leading edge portion 2E. A high frequency electrode 5 is arranged in the same manner as the first embodiment. Such configuration brings the same effects as the first embodiment.

The present disclosure relates to the subject matter contained in Japanese Patent Application No. P2006-258317, filed on Sep. 25, 2006, which is expressly incorporated herein by reference in its entirety.

What is claimed is:

1. A high frequency incision tool for an endoscope, comprising:
a flexible insulating tube configured to be inserted into and pulled out of a treatment tool insertion channel of the endoscope;
a conductive wire connectable with a high frequency power supply, the conductive wire being inserted and arranged in the flexible insulating tube;
a protruded portion formed to be partially protruded forward at a distal end portion of the flexible insulating tube, the protruded portion having a rounded leading edge; and
a high frequency electrode provided as a portion of the conductive wire that is exposed out of the flexible insulating tube, the high frequency electrode including at least a portion, closest to the leading edge of the protruded portion, which is substantially perpendicular to a virtual plane including therein an axis line of the flexible insulating tube and the leading edge of the protruded portion.

2. The high frequency incision tool according to claim 1,
wherein the high frequency electrode is arranged at a first half portion of the distal end portion of the flexible insulating tube opposite a second half portion that includes the rounded leading edge of the protruded portion when viewed from a distal end side of the flexible insulating tube.

3. The high frequency incision tool according to claim 1,
wherein the protruded portion is formed in a slantwise-cut shape with the rounded leading edge.

4. The high frequency incision tool according to claim 1,
wherein the protruded portion is formed to partially protrude in a tongue shape with the rounded leading edge.

5. The high frequency incision tool according to claim 2, further comprising a pair of openings arranged in a circumferential direction on an outer circumferential surface of the distal end portion of the flexible insulating tube, the pair of openings being configured such that the conductive wire can run therethrough,
wherein the high frequency electrode is exposed out of the flexible insulating tube between the pair of openings and arranged along an outer circumferential surface of the first half portion of the distal end portion of the flexible insulating tube.

6. The high frequency incision tool according to claim 5,
wherein the pair of openings are formed symmetrically with respect to the virtual plane.

7. The high frequency incision tool according to claim 1, further comprising a pair of openings arranged in a circumferential direction on an outer circumferential surface of the distal end portion of the flexible insulating tube, the pair of openings being configured such that the conductive wire can run therethrough,
wherein the high frequency electrode is exposed out of the flexible insulating tube between the pair of openings and arranged along a distal end surface of the distal end portion of the flexible insulating tube.

8. The high frequency incision tool according to claim 7,
wherein the high frequency electrode is configured to be substantially perpendicular to the virtual plane over an entire length thereof.

9. The high frequency incision tool according to claim 7,
wherein the high frequency electrode is formed substantially U-shaped, the U-shaped electrode including a pair of first portions substantially parallel to the virtual plane and a second portion substantially perpendicular to the virtual plane between the pair of first portions.

10. The high frequency incision tool according to claim 7,
wherein the pair of openings are formed symmetrically with respect to the virtual plane.

11. The high frequency incision tool according to claim 1,
wherein the flexible insulating tube includes a first tube and a second tube, and
wherein the first tube is connected with the second tube to be rotatable around the axis line of the flexible insulating tube with respect to the second tube.
